(19) United States
(12) Patent Application Publication
(10) Pub. No.: US 2023/0301794 A1
(43) Pub. Date: Sep. 28, 2023

(54) FIRST METATARSAL HEMI-ARTHROPLASTY IMPLANT

(71) Applicant: Richard MARKS, Mobile, AL (US)

(72) Inventor: Richard MARKS, Mobile, AL (US)

(21) Appl. No.: 18/132,649

(22) Filed: Apr. 10, 2023

Related U.S. Application Data
(63) Continuation-in-part of application No. 16/463,891, filed on May 24, 2019.
(60) Provisional application No. 62/428,858, filed on Dec. 1, 2016.

(51) Int. Cl. A61F 2/42; A61B 17/72

(52) U.S. Cl. CPC A61F 2/4225; A61B 17/7291; A61F 2002/4233; A61F 2002/30299

(57) ABSTRACT

A metatarsal hemi-arthroplasty implant may include a convex outer surface overlaying a concave inner surface wherein the convex outer surface is shaped to replace an articular surface of a distal end of a metatarsus. The implant may further include a stem that is configured to be inserted into an intramedullary canal of the metatarsus. The stem may extend from the concave inner surface along a longitudinal axis of the implant, and may have an outward-facing surface disposed at a first angle relative to the longitudinal axis, at least one flute extending along the length, and at least one radial groove formed in the outward-facing surface. The implant may further include a plurality of rib members distributed about the stem member to connect the stem to the inner concave surface. Each of the rib members may have a distally-facing surface disposed at a second angle relative to the longitudinal axis.

### CROSS-REFERENCE TO RELATED APPLICATIONS

The present application is a continuation-in-part of U.S. Pat. Application No. 16/463,891, filed May 24, 2019 and entitled FIRST METATARSAL HEMI-ARTHROPLASTY IMPLANT, which is a U.S. National Stage Entry of PCT Application Serial No. PCT/US2017/063361, filed Nov. 28, 2017 and entitled FIRST METATARSAL HEMI-ARTHROPLASTY IMPLANT, which claims the benefit of U.S. Provisional Application Serial No. 62/428,858, filed Dec. 1, 2016 and entitled FIRST METATARSAL HEMI-ARTHROPLASTY IMPLANT. The foregoing are incorporated by reference as though set forth herein in their entirety.

### TECHNICAL FIELD

The present invention relates generally to the field of orthopedic and podiatric surgery. More particularly, the present invention relates to the treatment of arthritis of the big toe joint or limited dorsiflexion (hallux rigidus) of the first metatarsophalangeal (MTP) joint as well as surgical treatment of the lesser metatarsophalangeal joints, because of dorsal osteophyte impingement or treatment of arthritis of the first MTP and lesser MTP joints.

### BACKGROUND

Arthritis of the big toe is the most common arthritic condition of the foot and is second only to bunions (hallux valgus) as a condition associated with the big toe. The true cause of hallux rigidus is not known although, several risk factors such as an abnormally long or elevated first foot bone (metatarsal), differences in foot anatomy, prior traumatic injury to the big toe or family history are believed to be contributing factors. Hallux rigidus is typically diagnosed by physical examination of the joint by a physician. This examination includes manipulation of the metatarsophalangeal (MTP) joint and examination of the foot for evidence of bone spurs. X-rays may then be used to help understand the extent of joint degeneration and to show the location and size of bone spurs.

Treatment of hallux rigidus typically consists of non-surgical therapy that includes anti-inflammatory medications, heat or ice, orthotics and injections. Surgical options are determined by the failure of the non-surgical therapy and the extent of the arthritis located in the MTP joint. Common surgical options for hallux rigidus and arthritis of the first MTP joint include decompression (cheilectomy), partial joint resection (resection arthroplasty), fusion (arthrodesis) of the 1st MTP joint, joint replacement and hemi-arthroplasty (partial joint replacement). Cheilectomy involves shaving the bone spur to allow more room for the toe to bend. Resection arthroplasty involves the cleaning of the arthritis and bone spurs from the MTP joint, combined with resection of the base of the proximal phalanx and then sewing the tissue around the joint capsule. The presence of arthritic complaints may fail to alleviate patients’ symptoms following cheilectomy, and resection arthroplasty has an unacceptably high rate of failure, loss of function and secondary deformity. First MTP joint fusion limits motion of the joint, and many active individuals report significant limitations of activity post-fusion. Existing joint replacement and hemiarthroplasty products/devices for treatment of first MTP joint arthritis commonly disrupt the surrounding soft tissues, creating loss of normal function of the first MTP joint, and resulting abnormal biomechanics and gait. Excessive bone resection can result in shortening of the hallux, loosening of implant fixation and implant failure. Revision or replacement of a failed first MTP implant or hemiarthroplasty thus becomes more complex due to bone loss, change in joint length or configuration and disruption of the surrounding soft tissues. The lesser MTP joints may also be affected by arthritic changes, though to a lesser degree. Common treatment consists of either bone resection, or resculpting of the metatarsal heads with a rongeur or reamer. This treatment can result in shortening of the metatarsal and respective toe, or transfer metatarsalgia as a result of the shortening. Therefore, it is important to maintain the same or similar length of the toe and joint as it was prior to the surgery.

Examples of various hemi-arthroplasty (partial joint) implants are disclosed in U.S. Pat. Publication No. 20040230303A1 filed by Saunders, U.S. Pat. Publication No. 20080051912 filed by Hollawell and U.S. Pat. Publication No. 20120259419A1 filed by Brown et. al. In each of these devices, a generally centrally positioned stem or anchor is fixated into the bone and a generally concave bearing or contact surface is provided. U.S. Pat. No. 9,044,332 granted to Goswami et. al., discloses numerous configurations of implantable devices for replacing all or a portion of the MTP joint. U.S. Pat. Publication No. 20100262254 filed by Lawrence et. al., disclosed a MTP implant that is oriented at an angle between approximately 45 and 75 degrees relative to the bone engaging surface. Similarly, PCT Publication No. WO 2011090711A1 filed by Beckendorf et. al, discloses a resurfacing implant including a head having a convex outer surface overlaying a concave inner surface. The implant also includes a stem extending from the inner surface such that the edge of the head surrounds the stem and overhangs a portion of the stem leading up to the inner surface. The device disclosed in the Beckendorf et. al publication appears to be similar to the ENCOMPASS metatarsal resurfacing implant sold by Osteomed, Inc. of Addison, Tex., USA. U.S. Pat. Publication No. 20120215320A1 discloses an implant for metatarsal hemiarthroplasty having first and second surfaces wherein the concave surface may include a pair of projection members which extend into the articular head of the bone.

There is a need for a surgical alternative that alleviates arthritic symptoms, while maintaining normal anatomy, biomechanics and motion of the first MTP joint. Ideally, a resurfacing option would limit bone resection and maintain soft tissue integrity, while allowing for the option of joint fusion if the hemi-arthroplasty is not successful and a full joint revision is necessary. The metatarsal hemi-arthroplasty of the present invention maintains metatarsal bone stock and length, while maintaining the integrity of the sesamoid complex, plantar plate, and collateral ligaments of the affected MTP joint. The implant is available to users in multiple sizing options for use in the 1st MTP or lesser MTP joints, and can be converted to a fusion procedure of the 1st MTP joint without the need for structural bone grafting material if revision is required. Additionally, the use of a single stem member with one or more spike members reduces the impact to the metatarsal head of the patient and minimizes the amount of bone that needs to be removed for implant placement. Because the amount of bone that is removed is minimized, if the implant fails, it is easier for the surgeon to perform a full joint revision.

### SUMMARY

In some embodiments, an implant configured to replace an articular surface on a distal end of a metatarsus may include a convex outer surface overlaying a concave inner surface wherein the convex outer surface is shaped to replace an articular surface of a distal end of a metatarsus. The implant may further include a stem that is configured to be inserted into an intramedullary canal of the metatarsus. The stem may extend from the concave inner surface along a longitudinal axis of the implant, and have an outward-facing surface disposed at a first angle relative to the longitudinal axis; at least one flute extending along the length of the stem; and at least one radial groove formed in the outward-facing surface. The implant may further include a plurality of rib members distributed about the stem member to connect the stem to the inner concave surface, wherein each of the rib members may include a distally-facing surface disposed at a second angle relative to the longitudinal axis and the second angle may be greater than the first angle.

In other embodiments, an implant may include a second angle within a range of 35 degrees to 65 degrees.

In still other embodiments, an implant may include one or more rib members that may be configured to prevent rotation of an implant after implantation in a metatarsus.

In still other embodiments, an implant may include at least one flute extending along a length of a stem. The flute may be configured to limit rotational movement of an implant after implantation in a metatarsus.

In still other embodiments, an implant may include a first spike spaced apart from the stem and extending from the inner concave surface generally parallel to the longitudinal axis.

In still other embodiments, an implant may include a plurality of additional spikes that, together with a first spike, may be spaced apart from the stem and may extend from the inner concave surface generally parallel to the longitudinal axis.

In still other embodiments, an implant may include a plurality of spikes that may be radially distributed in an asymmetrical pattern.

In still other embodiments, an implant may include a first spike including one or more radial grooves, wherein each of the one or more radial grooves may be configured to resist removal or the spike from the metatarsus.

In still other embodiments, an implant may include spikes wherein a diameter of a spike proximal end may be less than a diameter of a spike distal end.

In still other embodiments, an implant may be configured such that a convex outer surface, a concave inner surface, and a stem cooperate to define a cannulation extending along the longitudinal axis; and the cannulation is configured to receive a guidewire to guide insertion of the implant onto the distal end of the metatarsus.

In still other embodiments, an implant may include one or more side surfaces.

In still other embodiments, an implant may include one or more side surfaces wherein the one or more side surfaces may include one or more notches, wherein each of the one or more notches may include undercut surfaces.

In still other embodiments, an implant may include a convex outer surface that may include a first radius and a second radius. Further, a first radius may be on the dorsal side of an implant and a second radius may be on the plantar side of an implant.

In still other embodiments, an implant may include a first thickness, between a convex outer surface and a concave inner surface on a dorsal side of an implant and a second thickness, between a convex outer surface and a concave inner surface of an implant. Further, a first thickness may be less than a second thickness.

In still other embodiments, an implant may be configured such that a first cross-sectional length of a stem proximal end may be greater than a second cross-sectional length of a stem distal end.

In still other embodiments, an implant configured to replace an articular surface on a distal end of a metatarsus may include a convex outer surface overlaying a concave inner surface wherein the convex outer surface is shaped to replace an articular surface. The implant may further include a stem that may be configured to be inserted into an intramedullary canal of a metatarsus. A stem may extend from the concave inner surface along a longitudinal axis of the implant, and may comprise at least one flute extending along a length of a stem; and at least one radial groove. An implant may further include a first spike spaced apart from a stem and extending from an inner concave surface generally parallel to a longitudinal axis.

In still other embodiments, an implant may further include a plurality of additional spikes that, together with a first spike, may be spaced apart from a stem, and may be radially distributed about a stem, and may extend from an inner concave surface generally parallel to the longitudinal axis.

In still other embodiments, an implant may include a first spike including one or more radial grooves, wherein each of the one or more radial grooves may be configured to resist removal or the spike from the metatarsus

In other embodiments, an implant configured to replace an articular surface on a distal end of a metatarsus may include a convex outer surface overlaying a concave inner surface wherein the convex outer surface is shaped to replace an articular surface. The implant may further include a stem that is configured to be inserted into an intramedullary canal of the metatarsus. A stem may extend from the concave inner surface along a longitudinal axis of the implant, and have at least one flute extending along the length of a stem; and at least one radial groove. The implant may further be configured such that a convex outer surface, a concave inner surface, and a stem may cooperate to define a cannulation extending along a longitudinal axis; and a cannulation may be configured to receive a guidewire to guide insertion of an implant onto the distal end of a metatarsus.

In still other embodiments, an implant may include a cannulation wherein an axis of a cannulation may be parallel and offset from a stem longitudinal axis.

### BRIEF DESCRIPTION OF THE DRAWINGS

Exemplary embodiments of the disclosure will become more fully apparent from the following description and appended claims, taken in conjunction with the accompanying drawings. Understanding that these drawings depict only exemplary embodiments and are, therefore, not to be considered limiting of the disclosure’s scope, the exemplary embodiments of the disclosure will be described with additional specificity and detail through use of the accompanying drawings in which:

FIG. 1 is a side perspective view of a portion of a foot and a metatarsal implant device having features of the present invention;

FIG. 2 is front perspective view showing the implant device installed on the head of the metatarsal bone;

FIG. 3 is a side perspective view of the implant device having features of the present invention;

FIG. 4 is a top perspective view of the implant device showing the head surface of the implant;

FIG. 5 is a rear view of an alternate form of the implant device showing the stem and spikes of the implant;

FIG. 6 is a partial cross-sectional view of the implant device shown in FIG. 5;

FIG. 7 is a cross sectional view of an alternate form of the implant device of the present invention;

FIG. 8A is a side view, partially in cross section, of an alternate embodiment of an implant device of the present invention;

FIG. 8B is a cross sectional view of the embodiment of FIG. 8A;

FIG. 9A is a bottom view of an alternate embodiment of the implant device of the present invention;

FIG. 9B is a cross sectional view of the embodiment of FIG. 9A;

FIG. 10A is a bottom view of another alternate embodiment of the implant of the present invention;

FIG. 10B is a cross sectional view of the embodiment of FIG. 10A.

FIG. 11A is a bottom perspective view of another alternate embodiment of the present invention;

FIG. 11B is a cross sectional view of the embodiment of FIG. 11A;

FIG. 11C is a side view of the embodiment of FIG. 11A

FIG. 12A is a bottom perspective view of another alternate embodiment of the present invention;

FIG. 12B is a side view of the embodiment of FIG. 12A;

FIG. 12C is a cross sectional view of the embodiment of FIG. 12A;

FIG. 13A is a perspective view of a metatarsal arthroplasty implant, according to an embodiment of the present disclosure;

FIG. 13B is a cross-sectional side view of the metatarsal arthroplasty implant of FIG. 13A; and

FIG. 14 is a side view of the metatarsal arthroplasty implant of FIG. 13A installed in a metatarsal bone.

FIG. 15A is a perspective view of a cannulated metatarsal arthroplasty implant, according to an embodiment of the present disclosure;

FIG. 15B is a cross-sectional side view of the cannulated metatarsal arthroplasty implant of FIG. 15A; and

FIG. 16 is a side view of the cannulated metatarsal arthroplasty implant of FIG. 15A installed in a metatarsal bone.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the Figures herein, could be arranged and designed in a wide variety of different configurations. Thus, the following more detailed description of the embodiments of the apparatus and method, as represented in the figures, is not intended to limit the scope of the present disclosure, as claimed in this or any other application claiming priority to this application, but is merely representative of exemplary embodiments of the present disclosure.

The phrases “connected to,” “coupled to,” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature is able to pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

The first metatarsal hemi-arthroplasty system of the present invention includes implants and instrumentation to perform a resurfacing procedure of the first MTP or lesser MTP joints. The system includes instrumentation to assist in the placement of the implant, including but not limited to instrumentation to guide the implant to the desired location. Such instrumentation may include reamers, routers, rasps, broaches, saws, guide pins and guide wires as well as other placement or guidance tools specific to arthroplasty procedures. Additional tooling to establish pilot holes for the assistance of implant placement may be used.

The system also includes instrumentation for the preparation of the metatarsal head to receive the anatomy conserving implant. Bone preparation instrumentation may include, but is not limited to a reamer, saw, rasp and/or alternative bone preparation devices. Alternative bone preparation devices may include those devices used in arthroplasty procedures to resect tissue (soft and hard) to prepare the surgical site to receive the implant.

The system also includes instrumentation for implant sizing and instrumentation for inserting the implant into the metatarsal head. This may include implant embodiments for both manual insertion of the implant or implant insertion using instrumentation specific tools for implantation.

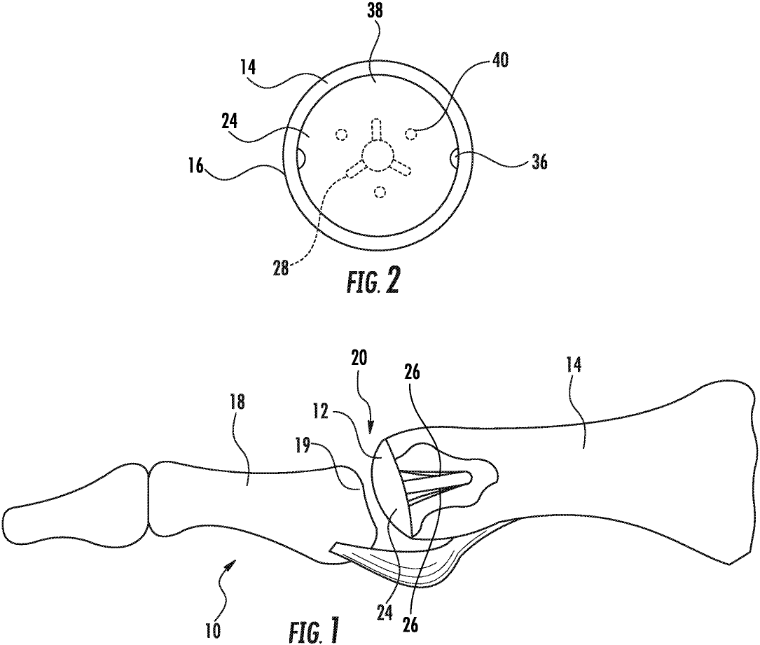

FIGS. 1 and 2 show the foot 10 of a patient having a metatarsal implant shown generally as component 12. The foot 10 includes a metatarsal bone 14 having a distal end 16 on which the implant 12 is attached. The foot 10 also includes a proximal phalanx bone 18 having a proximal end 19 which is positioned adjacent to the implant 12 and the distal end 16 of the metatarsal bone 14 to form the components of the metatarsal phalangeal joint 20.

The implant 12 is designed to be anatomy conserving and is comprised of a proximal stem 22 and a concave head 24. The proximal stem 22 of the implant 12 is implanted into a stem aperture 26 that is drilled into the distal end 16 of the metatarsal bone 14. As shown, the proximal stem 22 may have a generally conical or circular in cross sectional shape that decreases in width as it extends away from the concave head 24. In an alternate form of the present invention, the proximal stem 22 may include one or more flange like fin members 28 that extend laterally and taper inwardly from the proximal stem 22 to the end 23 thereof. As shown, the fin members 28 are wider and extend outwardly further near the proximal stem adjacent to the concave head 24 than the width of the fin members 28 adjected to the end 23 of the proximal stem 22. Alternatively, the proximal stem 22 may be comprised of a roughened or porous surface, a threaded surface or smooth surface.

The distal end of the implant 12 is a generally concave shaped head member 24. The head member 24 includes a smooth exterior surface 30that interfaces with the neighboring proximal phalanx bone 16 in the MTP joint 20. The concave head 24 is positioned generally adjacent to the proximal end 19 of the proximal phalanx bone 18 when the implant 12 is properly implanted onto the distal end 16 of the metatarsal bone 14. The head member 24 also includes an interior surface 32 (surface that interfaces with the metatarsal head) that may be comprised of a roughened, textured, or porous surface. Alternatively, a non-roughened surface or a non-porous surface may be used on the interior surface 32 of the implant 12 for an alternative implant embodiment. As shown, the exterior surface 30 and the interior surface 32 of the head member 24 preferably decrease in thickness from the center portion of the head member 24 to the periphery thereof to minimize the amount of bone that must be removed during the procedure to properly seat the implant 12. Also, as shown and described more fully below, the periphery of the head member 24 preferably includes one or more preferably laterally oriented notch members 36 to facilitate the removal of the implant if the doctor determines that the implant has failed, and a joint fusion or full joint implant is appropriate. Additionally, as shown, an etched member 38 is provided along the top edge thereof to assist in identifying the proper alignment of the implant 12 on the metatarsal bone 14.

The interior surface 32 of the head member 24 also includes a plurality of spike shaped members 40. The spike members 40 are spaced apart from the proximal stem 22 and each other. As shown in the drawings, the spike members 40 are preferably smaller and have a sharper profile than the proximal stem 22 to extend into the distal end 16 of the metatarsal bone 14. The spike members 40, in addition to the fin members 28 (if present), prevent rotational movement of the implant 12 once it is affixed to the distal end 16 of the metatarsal bone 14. FIGS. 2, 5 and 8-10 are illustrative of various alternate embodiments of the present invention. As shown, the implant 12 may include a proximal stem 22 that is tapered or of constant diameter. The stem may also have a plurality of fin members 28 arranged in a desired configuration such as three fins 28 arranged in a trident configuration preferably aligned at a 4, 8 and 12 clock orientations. The fin members may be of consistent width or may decrease in width near the end 23 of the proximal stern. One or more spike members 40 may also be included and in one configuration, the spike members 40 may be aligned at 2, 6 and 10 clock orientations. Alternately, two spike members 40 may be utilized at 2 and 10 o′clock configurations or a single spike may be aligned at a 6 or 12 o′clock configuration. The utilization of multiple fin members 28 and one or more spike members 40 provide added stability to the implant 12 to reduce the likelihood of rotational movement of the implant and therefore potential failure of the implant while minimizing the amount of bone removal required for proper implant placement.

The procedure for 1st MTP joint implantation is performed under regional anesthesia (ankle block) and intravenous sedation. The patient is placed supine on the operating room table, the foot and ankle are sterilely prepped to above the ankle. The limb is exsanguinated, and the procedure is performed typically with a tourniquet at the level of the ankle. A dorsal longitudinal incision is made starting at the distal ⅓ of the 1st metatarsal (MT), and extended distally to the proximal ⅓ of the proximal phalanx of the hallux. The EHL tendon is retracted laterally, and the capsule to the MTP joint sharply incised. Dissection is continued medially and laterally to allow exposure of the metatarsal head and base of the proximal phalanx. Sufficient exposure medially and laterally should allow full visualization of the inferior aspect of the 1st MT. Any bony eminence or osteophytes along the dorsal, medial and lateral aspect of the 1st MT are resected with a rongeur, chisel, and/or saw. Osteophytes at the dorsal base of the proximal phalanx are also resected with similar instrumentation. The sesamoid complex is mobilized with an elevator to maximize dorsiflexion of the 1st MTP joint. Although not described herein, a substantially similar surgical procedure is used for surgical procedures involving the lesser MTP joints.

A free reamer is used to initially determine the size of the MT head and the corresponding implant size, as well as to assist with orientation for the guide pin insertion. The guide pin is inserted under power to a depth corresponding to the laser-etched line on the guide pin, and the position of the guide pin is checked fluoroscopically in both the frontal and lateral planes. The surgeon then chooses between the standard reamer sizes of 14, 16, 18, 20, 22, or 24. If the size of the MT head is between sizes the surgeon uses the reamer size that is undersized to avoid impingement of the implant. The reamer has a window to assess the depth of bone resection. Markings are located on the guide pin for 1-6 mm resection. There is preferably a hard stop at 6 mm to prevent the guide pin from being inserted too far. Reaming is then performed to remove the cartilage and subchondral bone down to the bleeding cancellous bone.

The guide pin is then removed and a trial insertion device corresponding to the selected reamer size is inserted. The trial insertion device includes a small central peg and a top etched marking (for example, near 44) at the 12 o′clock measurement to ensure that the trial insertion device is properly aligned as shown in FIGS. 11 and 12. The joint is then moved through a full range of motion. 90 degrees of dorsiflexion is desired. If the joint is tight, additional mobilization of the sesamoids can be performed, or additional reaming of the metatarsal head is undertaken. If the joint appears loose, a +2 mm. head can be used as the trial insertion device.

If the implant includes one or more fin members 28, the guide pin is reinserted, and a fin template is placed over the guide pin. The template is oriented, so the laser-etched line sits at 12 o′clock relative to the MT head. The template also preferably has peripheral holes corresponding to the spike members 40. For example, if three spike members are provided, the peripheral holes are oriented at 2, 6 and 10 o′clock to provide for the stabilization of the template, as well as fixation points for the peripheral spike members 40 that serve as additional points of fixation of the implant. These holes are drilled with short 0.045″ Kirschner style wires. The number of peripheral holes will correspond to the number of spike members 40 that are to be used with the implant 12. A drop-in drill sleeve with inner and outer portions is then placed in the raised portion of the fin template. A tapered drill is inserted in the inner sleeve, and drilled to a preferred depth of about 14 mm to create the stem aperture 26. There is preferably a hard stop to prevent over-penetration of the tapered drill. The inner sleeve is then removed, and a broach for the fin members is placed over the guide pin, aligned with the dorsal laser-etched line, and impacted to create a space for the insertion of the fin members 28 of the implant 12 at a later step. The extractor is then used to remove the broach and the template is then removed from the MT head.

The final implant 12, which is separately packaged sterile, is then inserted with an impactor, taking care to orient the implant with a laser-etched line (for example, near 44) on the implant 12 and positioning the fin members 28 within the previously prepared fin slots, if present, and placement of the spike members 40 into the previously prepared holes. The hallux is placed through the entire range of motion to ensure there is no residual impingement. Fluoroscopic images are obtained in the frontal and lateral planes to verify accurate placement and sizing of the implant 12. If additional space preparation is needed or if the range of motion is limited, the implant 12 includes a pair of laterally spaced notch members 36 that may be accessed to assist in removing the implant 12. This process may be repeated until the surgeon is satisfied with the range of motion that is present in the metatarsal phalangeal joint 20. Intraoperative fluoroscopy may be repeatedly used to visualize the metatarsal shaft and confirm the final positioning of the implant.

Once the proper positioning and sizing of the implant is confirmed, the capsule is closed with 2-0 absorbable suture, 3-0 (or) 4-0 absorbable suture is used for subcutaneous tissues, and 4-0 nylon for skin closure. A compressive dressing is applied, and the foot placed in a short walking boot. Heel weight bearing is allowed, and range of motion exercises are initiated on day 2. Sutures are removed 10 days after surgery, patients are permitted to bear weight as tolerated, physical therapy initiated, and the patient weans out of the boot as tolerated.

The implant 12 of the present invention preferably includes a range of sizes such as 8, 9, 10, 11, or 12 mm for use with lesser metatarsal heads and 14, 16, 18, 20, 22, 24, 26 mm for use in the first metatarsal phalangeal joint 20. The implant may be made of a variety of standard implant materials. In the preferred form of the present invention, the implant may include a titanium head 24 with a plasma sprayed undersurface and a hydroxyapatite coated stem proximal 22. Alternately, the head may be made of a cobalt chrome with a hydroxyapatite coated stem or a trabecular metal stem. Yet another form of the present invention may include a porous bone contacting surface formed of a material such as a titanium based alloy with a powdered bed fusion process to form various pore sizes on a portion of the cap and the stem. Yet another form of the implant 12 of the present invention may be made using currently available 3D printing processes such that the desired pore sizes may be tightly controlled and tailored to various surface areas of the implant 12. Yet another form of the present implant 12 may include one or more recesses or pore surfaces that include bone growth promoting materials therein or thereon. The preferred pore sizes are in the range of between about 0.5 to 1 mm. As shown in FIG. 7, the preferred dimensions of the implant 12 may include a width of the base of the stem adjacent to the head 24 of between about 3.5 mm to 4.75 mm, more preferably between about 3.5 mm to 4.2 mm with a width of approximately 1.8 mm to about 2.4 mm at the end 23 of the proximal stem 22. The preferred length of the stem 22 is between about 12 mm to 14 mm. The preferred thickness of the head is between about 0.4 mm to 0.6 mm adjacent to the stem and preferably tapers to between about 0.25 mm to 0.035 mm at the periphery. Additionally, the thickness of the head adjacent to the proximal stem 22 is approximately twice the thickness of the head at the periphery. In the embodiments having the spike members, the diameter is preferably about 0.04 mm with a preferred length of approximately 3 mm to 7 mm, with a preferred length of about 3 mm such that the length of the spike members as compared to the length of the stem members is less than 50 percent and more preferably about 25 percent or less while the comparative diameter of the spike member 40 is preferably about 25 percent and more preferably about 10 percent or less than the diameter of the proximal stem 22. These ranges are approximate and will vary depending on the overall size of the implant and are preferably optimized to provide a stable and secure implant that will function for many years. As illustrated above, a preferred configuration of the stem includes a thickness at the distal tip that is approximately one-half of the thickness of the stem adjacent to the head.

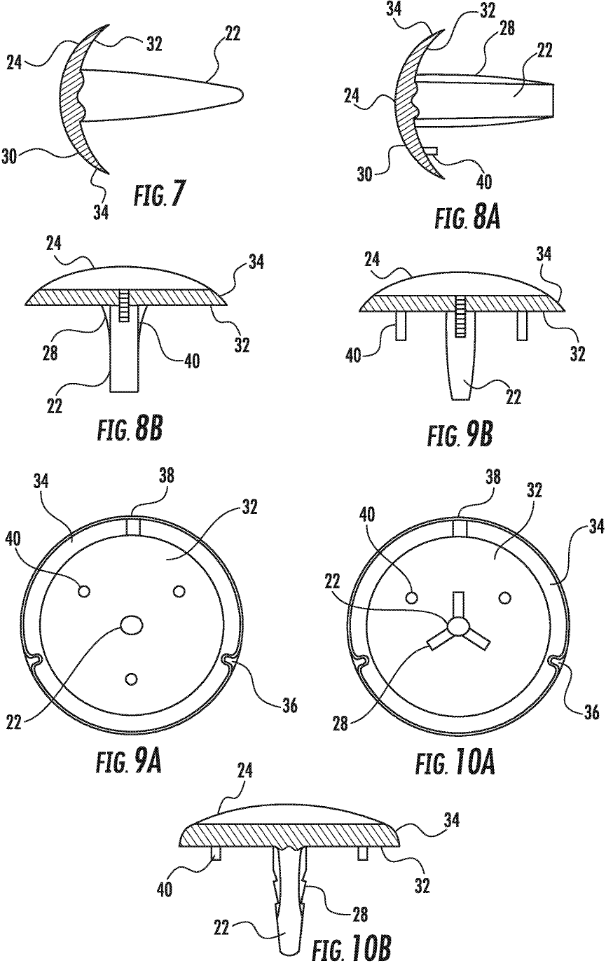

FIG. 7 is a side view, partially in cross section showing a tapered proximal stem 22 and illustrating the tapered thickness between the interior and exterior surfaces of the head 24. As shown in FIGS. 8A and 8B, the proximal stem 22 of the present invention may also include a consistent thickness between the base of the stem adjacent to the head to the end 23 of the proximal stem 22. As shown, this embodiment includes a single spike member preferably located at a 6 o′clock orientation. The proximal stem 22 of this embodiment may also include fin members 28 having a reduced outwardly extension the extend near the end 23 of the proximal stem 22 as described with the other embodiments. The preferred thickness is about 4.5 mm with a preferred length of about 14 mm.

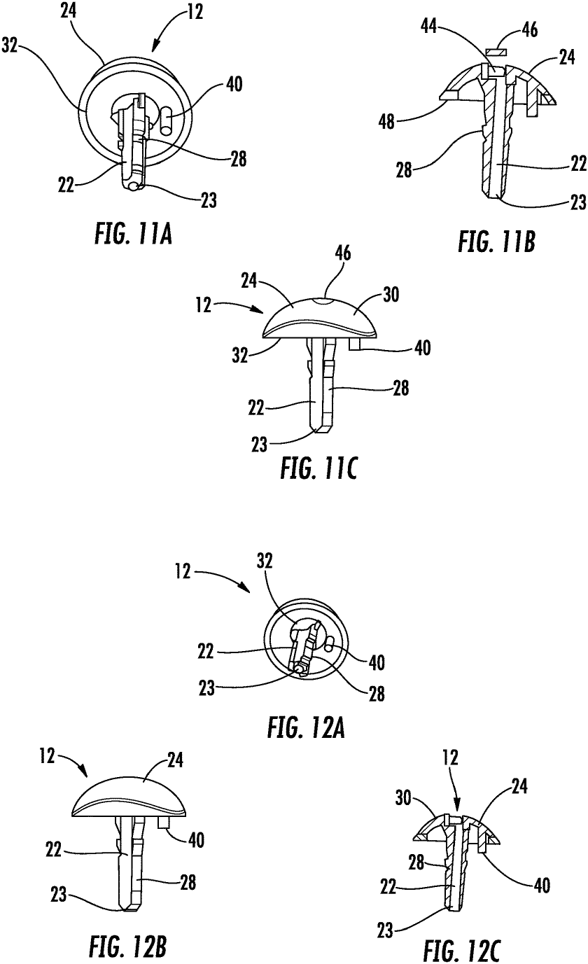

As shown in FIGS. 9A and B, the proximal stem 22 may be a tapered member without fin members. In this embodiment, three spike members 40 are preferably arranged at a preferred orientation of about 2, 6 and 10 and preferably have a greater length than is illustrated with the other embodiments. In this embodiment, the spike members 40 may include a length dimension between about 3 mm to 7 mm with a preferred length of about 6 mm. The proximal stem 22 of this embodiment may be thinner than the other embodiments with a preferred dimension in the range of about 3.5 to 4.2 mm and more preferably about 4 mm.

Another embodiment is shown in FIGS. 10A and 10B, wherein the proximal stem 22 is tapered and includes three fin members 28 oriented at a preferred orientation of about 4, 8 and 12. The fin members 28 of this embodiment are preferably cannulated to provide ridge type surfaces to resist the removal of the fin members 28 and proximal stem 22 from the distal end 16 of the metatarsal bone 14. In this embodiment, a pair of spike members 40 are also utilized. The spike members 40 are oriented at a preferred orientation of about 2 and 10 and are misaligned with the pair of notch members 36 located along the periphery of the implant 12.

Another embodiment is shown in FIGS. 11A, 11B and 11C, wherein the proximal stem 22 is tapered and includes three fin members 28 oriented at a preferred orientation of about 4, 8 and 12. The stem 22 of this embodiment is preferably cannulated so that a guide wire may be used during insertion. A guide wire may assist in inserting the implant in a preferred location within a distal end 16 of a metatarsal bone 14. In this embodiment, at least one spike member 40 is also utilized. The spike member 40 are oriented at a preferred orientation which is misaligned with fin members 28 and the pair of notch members 36 located along the periphery of the implant 12. In this embodiment, the greatest thickness between the inner surface 32 and exterior surface 34 of the head 24 of the implant is offset from the alignment of the proximal stem 22. The variation in thickness of the head 24 allows for the further alignment of the area of greater thickness with the desired location of the proximal end 19 of the proximal phalanx bone 18 to form the components of the surgically repaired metatarsal phalangeal joint 20 that more closely mimics the natural metatarsal phalangeal joint. As shown in FIGS. 11B and 11C, this embodiment may also include a threaded inset area 44 that allows the impactor to be threaded into the implant prior to the insertion of the implant 12 into the surgically prepared metatarsal bone. This connection enables the physician to apply direct force to the implant 12 as the implant 12 is inserted into the desired metatarsal bone via a secure connection between the impactor and implant. A removable cap 46 is also shown that may be threaded or also snapped into the inset area 44 to ensure that the outer surface of the head includes the desired exterior surface of the implant. Also shown with this embodiment, the implant 12 may include an outer ledge area 48, preferably oriented at about 9 and 3 to provide a surface area that may be grasped with a removal tool if it is desired to remove the implant from the joint.

FIGS. 12A, 12B and 12C are illustrative of another embodiment wherein the greatest thickness between the inner surface 32 and exterior surface 34 of the head 24 of the implant is offset from the alignment of the proximal stem 22. In this embodiment, the fin members are preferably cannulated and extend outwardly from the proximal stem 22 in a nearly uniform distance with none or only a small amount of reduced width between the extension of the fin member 28 adjacent to the head 24 and end 23 of the proximal stem. Additionally, the spike members 40 of this embodiment includes a greater length and thickness than the spike members 40 described with respect to the other embodiments described herein.

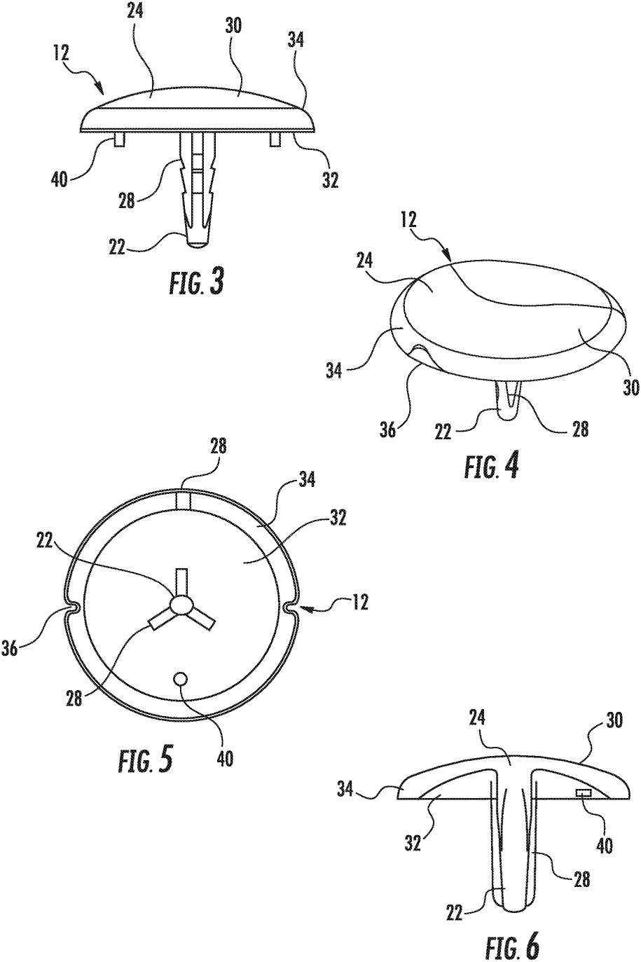

As disclosed herein, the implant 12 of the present invention may include 3 spike members 40 oriented at about 2, 6, and 10 o′clock (FIG. 2, FIGS. 9A and B and 12A and 12B); a single spike member 40 oriented at about 6 o′clock (FIG. 5, FIGS. 8A and B and FIGS. 11A, 11B and 11C); or 2 spike members 40 oriented and 2 and 10 (FIGS. 10A and B). The spike members 40 may also be oriented at different configurations without departing from the scope of the present invention. The implant 12 of the present invention may include various configurations of the proximal stem 22. As shown in FIGS. 2, 3-6 and 10A and B, the proximal stem 22 may be tapered and include a plurality of fin members 28 thereon. As shown, the fin members 28 are preferably tapered along the lengthwise dimension. As shown in FIGS. 7, and 9A and B, the proximal stem 22 may be a tapered member without fin members thereon. As shown in FIGS. 8A and B and FIGS. 12A and B, the proximal stem 22 may also have a substantially constant thickness. As with the other embodiments, the combination of the tapered or constant thickness proximal stem 22 with one or more spike members 40 resists rotational movement of the implant 12 when it is implanted and reduces the amount of bone that is harvested during implantation. Additionally, the stem 22 may be cannulated or non-cannulated.

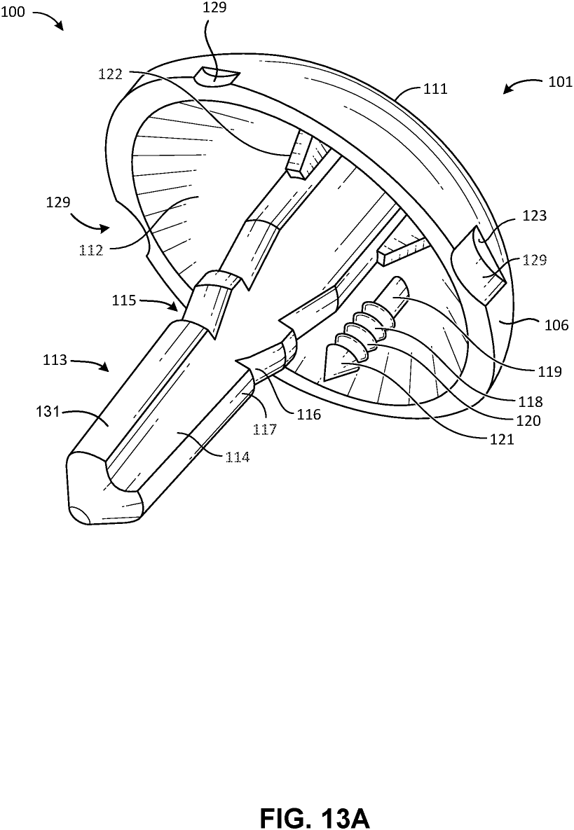

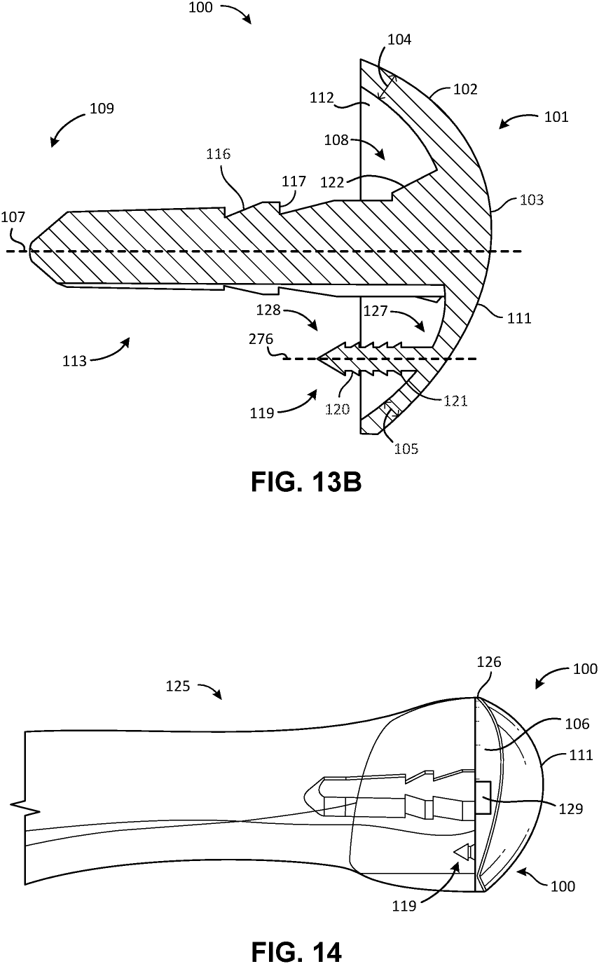

FIGS. 13A, 13B and 14 illustrate various views of a metatarsal arthroplasty implant or implant 100, according to an embodiment of the present disclosure. In some embodiments, the implant 100 may be designed to replace a natural joint engaging surface and articulate with a proximal phalanx. As the anatomy of patients may vary, the implant 100 may include a plurality of sizes, all with the same general profile and features.

In one embodiment, the implant 100 may have a convex articular surface or convex outer surface 111 and a concave bone-facing surface or concave inner surface 112. The convex outer surface 111 may be shaped to replace an articular surface of the distal end of a metatarsus. The concave inner surface 112 may couple with a central shaft or stem 113 extending therefrom. In some embodiments, a stem 113 may be configured to be inserted into an intramedullary canal of a metatarsus. Further, a stem 113 may include an outward facing surface 131 disposed at a first angle relative to a stem longitudinal axis 107. Further, a stem 113 may include a conical member with a constant thickness, or may decrease in thickness along the length thereof, and/or may include one or more longitudinal grooves or one or more flutes 114 extending along the length of the stem 113. The one or more flutes 114, extending along the length of a stem 113, may limit the rotational movement of the implant 100 after implantation in a metatarsus 125.

In some embodiments, the stem 113 may also include one or more barbs or radial grooves 115 that may be designed to prevent the implant 100 from backing out of the metatarsus 125 after implantation. Each of the one or more radial grooves 115 may be formed in the outward facing surface 131 and may have a first leading angle 116 and a second trailing angle 117. A stem 113 may be configured to be inserted into an intramedullary canal of a metatarsus 125.

In some embodiments, the first leading angle 116 may be within the range of 0° to 45° from a stem longitudinal axis 107. More specifically, the first leading angle 116 may be within a range of 10° to 35° from the stem longitudinal axis 107. Yet more specifically, the first leading angle 116 may be within a range of 20° to 25° from the stem longitudinal axis 107. Still more specifically, the first leading angle 116 may be approximately 22.5° from the stem longitudinal axis 107.

In some embodiments, the second trailing angle 117 may be within a range of 60° to 120° from the stem longitudinal axis 107. More specifically, the second trailing angle 117 may be within a range of 75° to 105° from the stem longitudinal axis 107. Yet more specifically, the second trailing angle 117 may be within a range of 85° to 95° from the stem longitudinal axis 107. Still more specifically, the second trailing angle 117 may be approximately 90° from the stem longitudinal axis 107.

Additionally, the concave inner surface 112 may include one or more spikes 119 protruding therefrom having spike proximal end 127 and a spike distal end 128. One spike 119 is shown in FIG. 13A, but in alternative embodiments, any number of spikes 119 may be present, and the spikes 119 may be distributed along the concave inner surface 112. Such distribution may or may not be along a radially symmetrical pattern. Each of the spikes 119 may be proximate the outer edge of the concave inner surface 112, closer to the stem 113, or positioned anywhere along the radius of the concave inner surface 112. This disclosure encompasses the use of 1, 2, 3, 4, 5, 6, 7, 8, 9, or more spikes 119. In some embodiments, a length of the one or more spikes 119 may be less than a length of a stem 113. A spike 119 may optionally have a generally circular cross-section such that the centerline or spike longitudinal axis 276 of a spike 119 may be parallel to the centerline or stem longitudinal axis 107 of a stem 113. In some embodiments, a spike longitudinal axis 276 of a spike 119 may be angled up to 10 degrees in any direction with respect to a stem longitudinal axis 107. Further, a spike 119 may include at least one spike barb or a plurality of radial grooves 118. Each of the plurality of radial grooves 118 may have a first leading angle 120 and a second trailing angle 121.

In some embodiments, the first leading angle 120 may be within the range of 10° to 60° from a spike longitudinal axis 276. More specifically, the first leading angle 120 may be within a range of 20° to 45° from the spike longitudinal axis 276. Yet more specifically, the first leading angle 120 may be within a range of 25° to 35° from the spike longitudinal axis 276. Still more specifically, the first leading angle 120 may be approximately 30° from the spike longitudinal axis 276.

In some embodiments, the second trailing angle 121 may be within a range of 60° to 120° from the spike longitudinal axis 276. More specifically, the second trailing angle 121 may be within a range of 75° to 105° from the spike longitudinal axis 276. Yet more specifically, the second trailing angle 121 may be within a range of 85° to 95° from the spike longitudinal axis 276. Still more specifically, the second trailing angle 121 may be approximately 90° from the spike longitudinal axis 276.

In some embodiments, the implant 100 may include one or more rib members 122 distributed about a stem 113 and connecting a stem 113 to a concave inner surface 112. The one or more rib members 122 may provide additional support for the stem 113 to help avoid bending of the stem 113 relative to the concave inner surface 112. The one or more rib members 122 may also engage with the metatarsus 125 as the implant 100 is implanted to help prevent rotation of the implant 100 after implantation in the metatarsus 125. Further, the one or more rib members 122 may comprise a distally facing surface disposed at an angle relative to a stem longitudinal axis 107. In some embodiments an angle at which one or more rib members 122 may be disposed relative to a stem longitudinal axis 107 may be within a range of 20° to 80° from a stem longitudinal axis 107. More specifically, an angle at which one or more rib members 122 may be disposed relative to a stem longitudinal axis 107 may be within a range of 35° to 65° from a stem longitudinal axis 107. In some embodiments, an angle at which one or more rib members 122 may be disposed relative to a stem longitudinal axis 107 may be greater than an angle at which an outward facing surface 131 may be disposed relative to a stem longitudinal axis 107.

In some embodiments, the implant 100 may include at least one side surface 106 with a notch or a plurality of notches 129 formed therein comprising undercut surfaces or undercuts 123.

FIG. 13B is a section view of the implant 100. In one embodiment, the implant 100 may have the convex outer surface 111 that may be comprised of a first radius 102 and a second radius 103 that may be generally similar to the first radius 102 or may be generally different than the first radius 102. In some embodiments, the articular member 101 or head of the implant 100 may include a first thickness 105 that may be less than a second thickness 104. In some embodiments, the first radius 102 may be on the dorsal side of the implant 100, and the second radius 103 may be on the plantar side. This may cause the adjoining phalanx to rotate about a tighter arc when moving dorsally of a horizontal plane through the axis of the metatarsus. Additionally, or alternatively, this variable radius may help maintain stability of the joint under the desired amount of tension from surrounding ligaments and other soft tissues. The radius of the convex outer surface 111 may be continuously variable between the first radius 102 and the second radius 103. In the alternative, there may be a more abrupt transition in radius, from the first radius 102 to the second radius 103. In alternative embodiments, other variable radius schemes may be used. For example, a smaller radius could exist on the plantar, rather than the dorsal, side. Variable radius may be used between the left and right sides of the convex outer surface 111.

FIG. 14 is a side view of the implant 100 fully seated in a metatarsus 125. In some embodiments, the rim 126 of the implant 100 may be generally aligned with the adjoining bony portion of the prepared metatarsal head, and the convex outer surface 111 may be positioned to articulate with the articulating surface of the phalanx (not shown) immediately distal to it.

In some embodiments, a metatarsal arthroplasty implant 100 may include an articular member 101, a stem 113 sized for insertion into a metatarsus 125, and one or more spikes 119 spaced apart from the stem 113 and sized for insertion into the metatarsus 125 adjacent the stem 113. The articular member 101 may include a convex outer surface 111, a concave inner surface 112 opposite the convex outer surface 111, and at least one side surface 106 intermediate the convex outer surface 111 and the concave inner surface 112. The stem 113 may include a stem longitudinal axis 107, a stem proximal end 108 coupled to the concave inner surface 112 of the articular member 101, and a stem distal end 109 extending away from the concave inner surface 112 of the articular member 101 along the stem longitudinal axis 107. The one or more spikes 119 may include a spike longitudinal axis 276, a spike proximal end 127 coupled to the concave inner surface 112 of the articular member 101, a spike distal end 128 extending away from the concave inner surface 112 of the articular member 101 along the spike longitudinal axis 276. In some embodiments, a diameter of a spike proximal end 127 may be less than a diameter of a spike distal end 128. One or more spikes 119 may further include one or more radial grooves 118 formed in the one or more spikes 119 and configured to resist removal of the one or more spikes 119 from the metatarsus 125.

In some embodiments, the articular member 101 may also include a plurality of notches 129 formed in the at least one side surface 106 with undercuts 123 extending below the convex outer surface 111 configured to facilitate installation and removal of the implant 100 from the metatarsus 125.

In some embodiments, a first cross-sectional length or diameter of the stem proximal end 108 may be greater than a second cross-sectional length or diameter of the stem distal end 109.

In some embodiments, the implant 100 may be intended to provide a replacement articulating surface of the metatarsal head and the implant 100 may be designed to articulate with the proximal phalanx. The implant 100 may be provided to a clinician in a single use sterilized package or packaging and/or may be provided in a package that requires sterilization prior to use. The packages may include trays specifically designed to accommodate the implant 100.

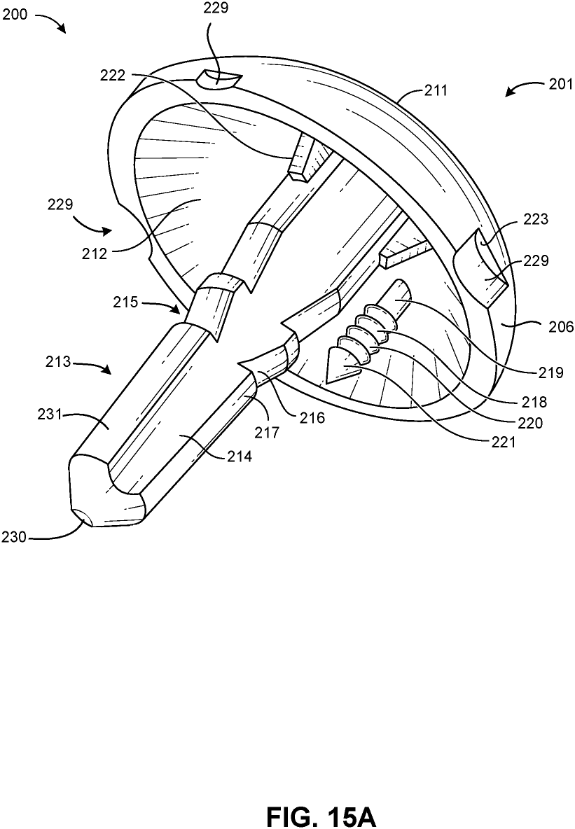

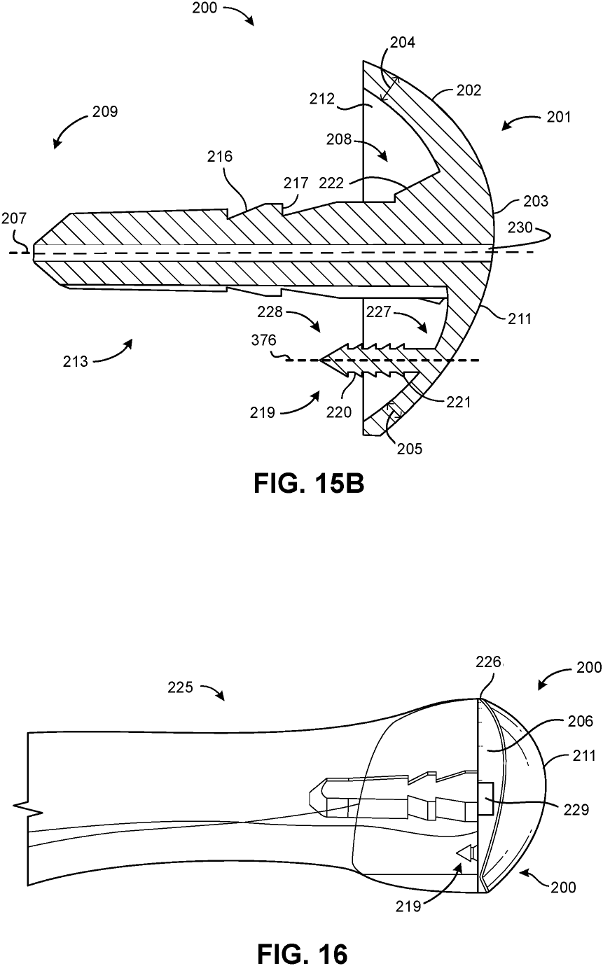

FIGS. 15A, 15B and 16 illustrate various views of a metatarsal arthroplasty implant or implant 200, according to an embodiment of the present disclosure. In some embodiments, the implant 200 may be designed to replace a natural joint engaging surface and articulate with a proximal phalanx. As the anatomy of patients may vary, the implant 200 may include a plurality of sizes, all with the same general profile and features.

In one embodiment, the implant 200 may have a convex articular surface or convex outer surface 211 and a concave bone-facing surface or concave inner surface 212. The convex outer surface 211 may be shaped to replace an articular surface of the distal end of a metatarsus. The concave inner surface 212 may couple with a cannulated central shaft or cannulated stem 213 extending therefrom. In some embodiments, a cannulated stem 213 may be configured to be inserted into an intramedullary canal of a metatarsus. Further, a cannulated stem 213 may include an outward facing surface 231 disposed at an angle relative to a stem longitudinal axis 207. Further, a cannulated stem 213 may include a conical member with a constant thickness, or may decrease in thickness along the length thereof, and/or may include one or more longitudinal grooves or one or more flutes 214 extending along the length of the cannulated stem 213. The one or more flutes 214 may limit the rotational movement of the implant 200 after implantation in a metatarsus 225.

In some embodiments, the convex outer surface 211, the concave inner surface 212, and the cannulated stem 213 may cooperate to define a cannulation 230 extending along the cannulated stem longitudinal axis 207. A cannulation 230 may be configured so that the axis of a cannulation is aligned with the cannulated stem longitudinal axis 207. Alternately, a cannulation 230 may be configured so that the axis of a cannulation is parallel with the cannulated stem longitudinal axis 207. Alternately, a cannulation 230 may be configured so that the axis of the cannulation is not parallel with the cannulated stem longitudinal axis 207. In some embodiments, a cannulation 230 may be generally cylindrical and may be configured with a generally consistent diameter. A cannulation 230 may be configured to receive a guide wire to guide insertion of an implant 200 onto a preferred location on the distal end of the metatarsus.

In some embodiments, the cannulated stem 213 may also include one or more barbs or radial grooves 215 that may be designed to prevent the implant 200 from backing out of the metatarsus 225 after implantation. Each of the one or more radial grooves 215 may be formed in the outward facing surface 231 and may have a first leading angle 216 and a second trailing angle 217. A stem 213 may be configured to be inserted into an intramedullary canal of a metatarsus.

In some embodiments, the first leading angle 216 may be within the range of 0° to 45° from a cannulated stem longitudinal axis 207. More specifically, the first leading angle 216 may be within a range of 10° to 35° from the cannulated stem longitudinal axis 207. Yet more specifically, the first leading angle 216 may be within a range of 20° to 25° from the cannulated stem longitudinal axis 207. Still more specifically, the first leading angle 216 may be approximately 22.5° from the cannulated stem longitudinal axis 207.

In some embodiments, the second trailing angle 217 may be within a range of 60° to 120° from the cannulated stem longitudinal axis 207. More specifically, the second trailing angle 217 may be within a range of 75° to 105° from the cannulated stem longitudinal axis 207. Yet more specifically, the second trailing angle 217 may be within a range of 85° to 95° from the cannulated stem longitudinal axis 207. Still more specifically, the second trailing angle 217 may be approximately 90° from the cannulated stem longitudinal axis 207.

Additionally, the concave inner surface 212 may include one or more spikes 219 protruding therefrom having spike proximal end 227 and a spike distal end 228. One spike 219 is shown in FIG. 15A, but in alternative embodiments, any number of spikes 219 may be present, and the spikes 219 may be distributed along the concave inner surface 212. Such distribution may or may not be along a radially symmetrical pattern. Each of the spikes 219 may be proximate the outer edge of the concave inner surface 212, closer to the cannulated stem 213, or positioned anywhere along the radius of the concave inner surface 212. This disclosure encompasses the use of 1, 2, 3, 4, 5, 6, 7, 8, 9, or more spikes 219. In some embodiments, a length of the one or more spikes 219 may be less than a length of a cannulated stem 213. The spikes 219 may optionally have a generally circular cross-section such that the centerline or spike longitudinal axis 376 of a spike 219 may be parallel to the centerline or cannulated stem longitudinal axis 207 of the cannulated stem 213. In some embodiments, a spike longitudinal axis 376 of a spike 219 may be angled up to 10 degrees in any direction with respect to a cannulated stem longitudinal axis 207. Further, a spike 219 may include at least one spike barb or a plurality of radial grooves 218. Each of the plurality of radial grooves 218 may have a first leading angle 220 and a second trailing angle 221.

In some embodiments, the first leading angle 220 may be within the range of 10° to 60° from a spike longitudinal axis 376. More specifically, the first leading angle 220 may be within a range of 20° to 45° from the spike longitudinal axis 376. Yet more specifically, the first leading angle 220 may be within a range of 25° to 35° from the spike longitudinal axis 376. Still more specifically, the first leading angle 220 may be approximately 30° from the spike longitudinal axis 376.

In some embodiments, the second trailing angle 221 may be within a range of 60° to 120° from the spike longitudinal axis 376. More specifically, the second trailing angle 221 may be within a range of 75° to 105° from the spike longitudinal axis 376. Yet more specifically, the second trailing angle 221 may be within a range of 85° to 95° from the spike longitudinal axis 376. Still more specifically, the second trailing angle 221 may be approximately 90° from the spike longitudinal axis 376.

In some embodiments, the implant 200 may include one or more rib members 222 distributed about a cannulated stem 213 and connecting a cannulated stem 213 to a concave inner surface 212. The one or more rib members 222 may provide additional support for the cannulated stem 213 to help avoid bending of the cannulated stem 213 relative to the concave inner surface 212. The one or more rib members 222 may also engage with the metatarsus 225 as the implant 200 is implanted to help prevent rotation of the implant 200 after implantation in the metatarsus 225. Further, the one or more rib members 222 may comprise a distally facing surface disposed at an angle relative to a stem longitudinal axis 207. In some embodiments an angle at which one or more rib members 222 may be disposed relative to a stem longitudinal axis 207 may be within a range of 20° to 80° from a stem longitudinal axis 207. More specifically, an angle at which one or more rib members 222 may be disposed relative to a stem longitudinal axis 207 may be within a range of 35° to 65° from a stem longitudinal axis 207. In some embodiments, an angle at which one or more rib members 222 may be disposed relative to a stem longitudinal axis 207 may be greater than an angle at which an outward facing surface 231 may be disposed relative to a stem longitudinal axis 207.

In some embodiments, the implant 200 may include at least one side surface 206 with a notch or a plurality of notches 229 formed therein comprising undercut surfaces or undercuts 223.

FIG. 15B is a section view of the implant 200. In one embodiment, the implant 200 may have the convex outer surface 211 that may be comprised of a first radius 202 and a second radius 203 that may be generally similar to the first radius 202 or may be generally different than the first radius 202. In some embodiments, the articular member 201 or head of the implant 200 may include a first thickness 205 that may be less than a second thickness 204. In some embodiments, the first radius 202 may be on the dorsal side of the implant 200, and the second radius 203 may be on the plantar side. This may cause the adjoining phalanx to rotate about a tighter arc when moving dorsally of a horizontal plane through the axis of the metatarsus. Additionally, or alternatively, this variable radius may help maintain stability of the joint under the desired amount of tension from surrounding ligaments and other soft tissues. The radius of the convex outer surface 211 may be continuously variable between the first radius 202 and the second radius 203. In the alternative, there may be a more abrupt transition in radius, from the first radius 202 to the second radius 203. In alternative embodiments, other variable radius schemes may be used. For example, a smaller radius could exist on the plantar, rather than the dorsal, side. Variable radius may be used between the left and right sides of the convex outer surface 211.

FIG. 16 is a side view of the implant 200 fully seated in a metatarsus 225. In some embodiments, the rim 226 of the implant 200 may be generally aligned with the adjoining bony portion of the prepared metatarsal head, and the convex outer surface 211 may be positioned to articulate with the articulating surface of the phalanx (not shown) immediately distal to it.

In some embodiments, a metatarsal arthroplasty implant 200 may include an articular member 201, a cannulated stem 213 sized for insertion into a metatarsus 225, and one or more spikes 219 spaced apart from the cannulated stem 213 and sized for insertion into the metatarsus 225 adjacent the cannulated stem 213. The articular member 201 may include a convex outer surface 211, a concave inner surface 212 opposite the convex outer surface 211, and at least one side surface 206 intermediate the convex outer surface 211 and the concave inner surface 212. The cannulated stem 213 may include a cannulated stem longitudinal axis 207, a stem proximal end 208 coupled to the concave inner surface 212 of the articular member 201, and a stem distal end 209 extending away from the concave inner surface 212 of the articular member 201 along the cannulated stem longitudinal axis 207. The one or more spikes 219 may include a spike longitudinal axis 376, a spike proximal end 227 coupled to the concave inner surface 212 of the articular member 201, a spike distal end 228 extending away from the concave inner surface 212 of the articular member 201 along the spike longitudinal axis 376. In some embodiments, a diameter of a spike proximal end 227 may be less than a diameter of a spike distal end 228. One or more spikes 219 may further include one or more radial grooves 218 formed in the one or more spikes 219 and configured to resist removal of the one or more spikes 219 from the metatarsus 225.

In some embodiments, the articular member 201 may also include a plurality of notches 229 formed in the at least one side surface 206 with undercuts 223 extending below the convex outer surface 211 configured to facilitate installation and removal of the implant 200 from the metatarsus 225.

In some embodiments, a first cross-sectional length or diameter of the stem proximal end 208 may be greater than a second cross-sectional length or diameter of the stem distal end 209.

In some embodiments, the implant 200 may be intended to provide a replacement articulating surface of the metatarsal head and the implant 200 may be designed to articulate with the proximal phalanx. The implant 200 may be provided to a clinician in a single use sterilized package or packaging and/or may be provided in a package that requires sterilization prior to use. The packages may include trays specifically designed to accommodate the implant 200.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily referring to the same embodiment.

Similarly, it will be appreciated that in the above description of embodiments, various features are sometimes grouped together in a single embodiment, figure, or description for the purpose of streamlining the disclosure. This method of disclosure, however, is not to be interpreted as reflecting an intention that any claim in this or any application claiming priority to this application require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Only elements recited in means-plus-function format are intended to be construed in accordance with 35 U.S.C. §112 Para. 6. It will be apparent to those having skill in the art that changes may be made to the details of the above-described embodiments without departing from the underlying principles of the disclosure.

While specific embodiments and applications of the present disclosure have been illustrated and described, it is to be understood that the disclosure is not limited to the precise configuration and components disclosed herein. Various modifications, changes, and variations which will be apparent to those skilled in the art may be made in the arrangement, operation, and details of the methods and systems of the present disclosure herein without departing from the spirit and scope of the disclosure.

What is claimed is:

1. An implant configured to replace an articular surface on a distal end of a metatarsus, the implant comprising:
a convex outer surface overlaying a concave inner surface, wherein the convex outer surface is shaped to replace the articular surface;
a stem configured to be inserted into an intramedullary canal of the metatarsus, the stem extending from the concave inner surface along a longitudinal axis of the implant, the stem comprising:
an outward-facing surface disposed at a first angle relative to the longitudinal axis;
at least one flute extending along a length of the stem; and
at least one radial groove formed in the outward-facing surface; and
a plurality of rib members distributed about the stem to connect the stem to the inner concave surface; wherein:
each of the rib members comprises a distally-facing surface disposed at a second angle relative to the longitudinal axis; and
the second angle is greater than the first angle.

2. The implant of claim 1, wherein the second angle is within a range of 35 degrees to 65 degrees.

3. The implant of claim 1, wherein the rib members are configured to prevent rotation of the implant after implantation in the metatarsus.

4. The implant of claim 1, wherein the rib members are configured to provide structural support between the concave inner surface and the stem.

5. The implant of claim 1, further comprising a first spike spaced apart from the stem and extending from the inner concave surface generally parallel to the longitudinal axis.

6. The implant of claim 5, further comprising a plurality of additional spikes that, together with the first spike, are spaced apart from the stem, are radially distributed about the stem, and extend from the inner concave surface generally parallel to the longitudinal axis.

7. The implant of claim 5, wherein the first spike is positioned about two-thirds of a distance between the longitudinal axis and a side surface of the convex outer surface.

8. The implant of claim 5, wherein:
the first spike comprises one or more radial grooves; and
each of the one or more radial grooves comprises a barb shape configured to resist removal of the first spike from the metatarsus.

9. The implant of claim 5, wherein the first spike is generally straight and comprises a sharp distal tip.

10. The implant of claim 1, wherein:
the convex outer surface, the concave inner surface, and the stem cooperate to define a cannulation extending along the longitudinal axis; and the cannulation is configured to receive a guidewire to guide insertion of the implant onto the distal end of the metatarsus.

11. The implant of claim 1, wherein the convex outer surface comprises an asymmetrical shape such that the longitudinal axis does not pass through a location on the convex outer surface that is furthest from a distal end of the stem.

12. The implant of claim 11, wherein:
The convex outer surface comprises one or more side surfaces comprising one or more notches; and:
each of the one or more notches comprises an undercut surface configured to facilitate retention of the implant with an instrument.

13. The implant of claim 1, wherein:
the convex outer surface comprises a first radius and a second radius different from the first radius; and
the first radius is on a dorsal side of the implant and the second radius is on a plantar side of the implant.

14. The implant of claim 1, further comprising
a first thickness, between the convex outer surface and the concave inner surface on a dorsal side of the implant;
a second thickness, between the convex outer surface and the concave inner surface on a plantar side of the implant; and
the first thickness is less than the second thickness.

15. An implant configured to replace an articular surface on a distal end of a metatarsus, the implant comprising:
a convex outer surface overlaying a concave inner surface, wherein the convex outer surface is shaped to replace the articular surface;
a stem configured to be inserted into an intramedullary canal of the metatarsus, the stem extending from the concave inner surface along a longitudinal axis of the implant, the stem comprising:
at least one flute extending along a length of the stem; and
at least one radial groove; and
a first spike spaced apart from the stem and extending from the inner concave surface generally parallel to the longitudinal axis.

16. The implant of claim 15, further comprising a plurality of additional spikes that, together with the first spike, are spaced apart from the stem and radially distributed about the stem, and extend from the inner concave surface generally parallel to the longitudinal axis.

17. The implant of claim 15, wherein:
the first spike comprises one or more radial grooves; and
each of the one or more radial grooves comprises a barb shape configured to resist removal of the first spike from the metatarsus.

18. An implant configured to replace an articular surface on a distal end of a metatarsus, the implant comprising:
a convex outer surface overlaying a concave inner surface, wherein the convex outer surface is shaped to replace the articular surface; and
a stem configured to be inserted into an intramedullary canal of the metatarsus, the stem extending from the concave inner surface along a longitudinal axis of the implant, the stem comprising:
at least one flute extending along a length of the stem; and
at least one radial groove; wherein:
the convex outer surface, the concave inner surface, and the stem cooperate to define a cannulation extending along the longitudinal axis; and
the cannulation is configured to receive a guidewire to guide insertion of the implant onto the distal end of the metatarsus.

19. The implant of claim 18, wherein an axis of the cannulation is parallel to and offset from the longitudinal axis.

20. The implant of claim 18, wherein the convex outer surface comprises an asymmetrical shape such that the cannulation does not pass through a location on the convex outer surface that is furthest from a distal end of the stem.
